SEQUENCE LISTING (1 sequence)

SEQ ID NO: 1 - protein, 9 aa, H. influenzae
GILGFVFTL

(19) United States
(12) Patent Application Publication
(10) Pub. No.: US 2007/0237771 A1
(43) Pub. Date: Oct. 11, 2007

(54) Multicomponent conjugates which bind to target molecules and stimulate T cell lysis

(76) Inventors: Alena Donda, Epalinges (CH); Jean-Pierre Mach, Epalinges (CH); Valerie Cesson, Epalinges (CH); Bruno Robert, Epalinges (CH); Pedro Romero, Epalinges (CH); Immanuel Leuscher, Gollion (CH); Philippe Guillaume, Lausanne (CH)

Correspondence Address: FULBRIGHT & JAWORSKI, LLP, 666 FIFTH AVE, NEW YORK, NY, 10103-3198, US

(21) Appl. No.: 11/804,945

(22) Filed: May 21, 2007

Related U.S. Application Data
(63) Continuation of application No. 10/632,663, filed on Aug. 1, 2003.
Application No. 10/632,663 is a continuation-in-part of application No. 10/276,764, filed on Feb. 10, 2003.

(51) Int. Cl. A61K 39/395; A61K 38/16; A61P 35/00; C07K 14/00; C07K 16/00

(52) U.S. Cl. 424/138.1; 424/130.1; 514/12; 530/350; 530/387.1

(57) ABSTRACT

The invention relates to immunoconjugates of formula:
A-B—(C)ₙ where B may be present or absent, A is a specific binding protein such as an antibody or an antibody binding fragment, or a ligand binding to a receptor present on target cells B comprises at least one molecule to which “A” and “C” bind, such as an avidin/streptavidin complex, “C” is an MHC molecule, and “n” is a whole number ranging from 1 to 10. The conjugates provide the exquisite binding specificity of antibodies, combined with an ability to stimulate cytotoxic T cells to identify and to destroy cells on which the conjugate is bound and oligomerized. The conjugates are useful both therapeutically and diagnostically.

### RELATED APPLICATION

This application is a continuation in part of Ser. No. 10/276,764 filed Nov. 19, 2002, incorporated by reference in its entirety.

### FIELD OF THE INVENTION

This invention relates to conjugates, or fusion proteins, which comprise a specific, cell surface binding molecule, and an antigenic complex of an MHC molecule and a peptide, as well as uses of these. Such constructs bind to target cells, leading to activation of T lymphocytes, and induction of cytotoxicity, and serve as useful therapeutic agents.

### BACKGROUND AND PRIOR ART

Antibodies are high molecular weight proteins which recognize and bind specifically to molecules, such as foreign molecules (e.g., proteins, glycoproteins, lipoproteins, etc.), which are sometimes referred to as antigens, or markers. The term “marker” is used frequently when the antibody target is found on the surface of a subpopulation of cells, such as tumor cells, or cells bearing one or more differentiation antigens, also called “clusters of differentiation” or “CDs.” Antibodies bind to specific epitopes formed by the target molecule. While antibodies are known for their excellent binding and targeting ability, they are not particularly efficient at killing target cells to which they bind.

Antibodies represent just one facet of the immune system. T lymphocytes are cells which have surface receptors that are capable of recognizing, e.g., viral or tumor antigens, only in the form of short peptides, presented within the groove of so-called “major histocompatibility complexes” or “MHC”s on the surface of cells. The recognition of and binding to peptides associated with MHC on the surface of target cells leads to the activation of specific T lymphocytes, and often to the lysis of the cells expressing the specific MHC peptide complex. This is a very efficient killing mechanism; however, sometimes virally infected cells or, more frequently, tumor cells, escape the T lymphocyte attack by, e.g., deleting expression of molecules that are a part of the MHCs, and hence their expression on cell surfaces.

There are two classes of MHC molecules, i.e., “Class I” and “Class II” MHC molecules. The first class is expressed on the surface of most human cells, while constitutive expression of Class II molecules is limited for the most part to B lymphocytes, dendritic cells, and macrophages. These three cell types function as “antigen presenting cells.”

Structurally, MHC Class I molecules consist of three components: (i) a heavy chain with a molecular weight of about 50 kilodaltons, (ii) a light, non-polymorphic chain, referred to as beta-2 microglobulin, “beta2M”, or “β2M” and (iii) a peptide which generally consists of 8-10 amino acids which lies in a specific groove made by the heavy chain N terminal domain of MHC. The first item, i.e., the heavy chain, exhibits genetic polymorphism at its extracellular N-terminus, and a non-polymqrphic, partially intracellular C-terminus. The third item, i.e., the peptide, varies, depending upon the nature of the polymorphism in (i). When these three elements form an MHC presented on cell surfaces, the complex is referred to as a T cell antigen, and CD8⁺ T lymphocytes with appropriate receptors bind to them and act as described supra. See Townsend, et al., Ann. Rev. Immunol., 7:601-24 (1989), incorporated by reference, for a discussion of this structure.

The MHC Class II molecules which consist of two chains, α and β, of similar size, present longer peptides, 15-25 amino acids long, to CD4+ T lymphocytes. Soluble, recombinant Class I and II MHCs have been expressed in bacteria and insect cells, respectively (Garboczi, et al., Proc. Natl. Acad Sci USA, 89:3429-33 (1992), Stern, et al., Nature, 368:215-21 (1994) incorporated by reference). Further, artificial forms of recombinant MHC Class I have been synthesized, which consist of a single chain containing all three of the aforementioned elements. These molecules were synthesized via using genes encoding a fusion protein. These molecules retained their capacity to be recognized by T lymphocytes. See Mottez, et al., J. Exp. Med, 181:493-502 (1995), incorporated by reference.

Recently, recombinant MHC Class I molecules which contain 15 amino acid sequences at their C terminus that allow the site specific coupling of biotin on a lysine residue by the BirA enzyme, have been synthesized. See Schatz, et al., Biotechnology, 11:1138-43 (1993) (1996), incorporated by reference. These molecules can be biotinylated at their C-terminal end, which permits tetramerization via binding of biotin molecules to the four binding sites on avidin or streptavidin. These tetramers bind with higher affinity to T lymphocytes expressing specific receptors, thanks to multiple cooperative bonds. See Altman, et al., Science, 274:94-6 (1996) incorporated by reference. In addition, if the streptavidin or avidin molecule used is labelled with, e.g., a fluorescent molecule, such as phycoerythrin, the tetramers can be used in vitro in order to characterize T cells specific for a given antigenic peptide via, e.g., flow cyofluorimetry. These complexes have been used to characterize the afferent arm of the T lymphocyte response, but not for the study of their effector properties.

In the field of cancer immunotherapy, one approach that has generated great interest is the systemic injection of high affinity monoclonal antibodies directed against cell surface tumor associated antigens. This approach has resulted in statistically significant remission in β cell lymphoma and breast cancer. See McLaughlin, et al., J. Clin. Oncol., 16(8):2825-2833 (1998); and Cobleigh, et al., J. Clin. Oncol., 17(9):2639-2648 (1999).

In spite of the encouraging results reported supra, the percentage of complete human remission resulting from repeated injection of the monoclonal antibodies, as the single therapeutic approach, remains low. It has been learned, for example, from injection of radiolabeled monoclonal antibodies, that the treated antigens need not be tumor specific, as long as they are easily accessible, and overexpressed on cancer cells, as compared to normal cells. See Welt, et al., J. Clin. Oncol., 12(8):1561-1571 (1994); Delaloye, et al., J. Clin. Invest., 77(1):301-311 (1986), dealing with carcinoembryonic antigen (CEA), and A33. Further, it is known that treatment with anti-CD20 monoclonal antibodies, which react with both normal and malignant cells, is clinically beneficial and the monoclonals are well tolerated by the subjects.

A second approach to cancer immunotherapy involves active immunization of a subject with one or more antigens that are known to be recognized by and to activate T lymphocytes. The MAGE group of antigens, as well as NY-ESO-1 are examples of these. See Boon, et al., Annu. Rev. Immunol., 12:337-365 (1994); Chen, et al., Proc. Natl. Acad. Sci. USA, 94(5):1914-1918 (1997). These antigens belong to the so-called “canter testis antigen family,” since, their expression is limited to tumor cells, and spermatogenetic cells from the testis. See Old, et al., Cancer Immun., 1:1 (2001). Positive results have been seen, following vaccination, via MHC-tetramer staining of the T lymphocyte response of the patients. Promising results need to be considered together with low percentage of tumor remission, as is reported by Marchand, et al., Int. J. Cancer, 80(2):219-230 (1999); Lee, et al., Nat. Med., 5(6):677-685 (1999), as well as a low correlation between clinical and I Lymphocyte responses to vaccination. See Lee, et al., supra, Pharmiani, et al., Cancer Immun., 2:6 (2002); Rosenberg, et al., Nat. Med., 4(3):321-327 (1998). There is also a risk, as discussed by Ferrone, et al., Immunol. Today, 16(10):487-494 (1995), that HLA-lost tumor cell variants can be selected, via this approach.

It has been shown, by Ogg, et al., Br. J. Cancer, 82(5):1058-1062 (2000); and Robert, et al., Eur. J. Immunol., 30(11):3165-3170 (2000), both of which are incorporated by reference, that biotinylated, MHC/peptides that have been multimerized on streptavidin, and coupled to tumor cell specific antibodies, induce T lymphocyte mediated lysis of cells in vitro. Robert, et al., Cancer Immun., 1:2 (2001), also incorporated by reference, took this work further by showing that monomeric HLA-A2/Flu matrix peptides, when directly coupled to a specific Fab′ fragment, were active in killing various human tumor cell lines. Indeed, it has been shown that any of anti-CEA, ErbB-2, or CD-20, coupled to HLA-A2/flu matrix peptides, oligomerize on human tumor cell lines which present the relevant tumor associated antigen, and induce lysis, in vitro, by flu matrix peptide specific, HLA-A2 restricted T cells. See Robert, et al., supra. Recently, it has been shown, that in vitro preincubation and coating of tumor cells with MHC/peptide conjugates, which were then grafted into, immunodeficient mice, followed by coinjection of relevant activated T lymphocytes specifically prevented tumor development. See, Savage, et al., Int. J. Cancer, 98(4):561-566 (2002); and Lev, et al., J. Immunol., 169(6):2988-2996 (2002). Such an approach; however, cannot be considered to be a complete in vivo model.

One aspect of the invention relates to conjugates which combine the high binding specificity of specific, cell surface binding molecules, such as antibodies for their targets, or ligands for various receptors, and the capacity of MHC/peptide complexes, oligomerized on target cells, to stimulate specific cytolytic T lymphocytes.

It is a further aspect of the invention to present conjugates of Fab′ fragments, MHC molecules and peptides, which provide a real link between the antigen recognition property of antibodies which bind to epitopes on large, native molecules, and the recognition properties of T cell receptors, which bind to antigenic, short peptides expressed in MHC complexes.

It is a further aspect of the invention to provide a method for eliminating target cells by contacting these with conjugates of the type described supra and inducing their lysis by T lymphocytes.

It is a further aspect of the invention to provide conjugates which include the specific binding proteins coupled to MHC, described supra, is directed against an antigen or marker expressed on an antigen presenting cell. When oligomerized on the surface of antigen presenting cells, the MHC/peptide complexes stimulate T cells, in a manner mimicking the vaccination effect.

Multimeric complexes of streptavidin, biotin, and MHC molecules are known from the art. See, in this regard, Dunbar, et al., Tumor Immunol., 92(12):3.3 (1997); Altman et al., supra, and PCT publication WO 99/50637, to Romero, et al.; all of which are incorporated by reference. Apart from describing the general concept of streptavidin-biotin-MHC multimeric complexes, the PCT publication describes how these can be adapted to the class of molecules referred to as tumor rejection antigens, or “TRAs.” More information on TRAs can be found in, e.g., U.S. Pat. Nos. 6,025,470; 5,554,724; 5,554,506, and 5,487,974, all of which are incorporated by reference. The concept of the tumor rejection antigen is described in U.S. Pat. No. 5,342,774, also incorporated by reference. There is a vast patent literature on these molecules, and the specific members of the family of MHC molecules of which they are a part.

None of these references suggest, however, that the monomeric MHC/peptide complexes could be directly conjugated or fused to binding proteins, such as antibodies or binding fragments of antibodies directed against markers abundantly expressed on the surface of target cells, can induce the oligomerization of MHC complexes on target cells resulting in optimal recognition by T lymphocytes. Such bifunctional conjugates, which are described herein, are useful in targeting specific cells, as well as in destroying these targeted cells, via the intervention of cytolytic T cells.

Furthermore, when the target cells of such conjugates containing a binding protein and MHC class I or class II MHC/peptide complexes belong to the category of antigen presenting cells, such as dendritic cells or B lymphocytes, activation of specific CD8 or CD4 T lymphocytes can be induced.

The examples which follow also demonstrate that one can use the antibody-MHC-peptide complexes in vivo as they effectively eradicate cancer cells in an in vivo model. The data which follows represent the first successful showing of efficacy of complexes as described herein, in an in vivo model.

### BRIEF DESCRIPTION OF THE FIGURES

FIG. 1 presents flow cytometry analysis demonstrating the specific coating of anti-tumor Fab-HLA-A2/flu conjugates on the surface of HLA-A2 negative tumor cells, using a FITC-labeled anti-HLA-A2 mAb.

FIGS. 2A-2C shows results of induction of specific lysis, measured by a⁵¹Cr release assay, of different types of cancer cells preincubated with a constant amount of 2 μg/ml of said bifunctional conjugates in accordance with the invention, with a titration of specific cytotoxic T lymphocyte at effector to target cell ratio, ranging from 0.1 to 30/1. FIG. 2A shows results using anti-CEA Fab′ conjugates, 2B shows results obtained using anti-HER2 Fab′ containing conjugates, and 2C shows results using anti-CD20 Fab′ conjugates.

FIGS. 3A-C shows results of induction of specific lysis of different types of cancer cells preincubated with different amounts of said bifunctional conjugates in accordance with the invention, ranging from 10−¹ to 10³ ng/ml in presence of a constant effector to target cell ratio of 10/1. The panels parallel those of FIG. 2.

FIG. 4 schematically describes the mechanism, by which the described Fab-MHC conjugates can induce very efficient target cell killing by specific cytotoxic T lymphocytes, through oligomerization of the conjugate on the target cell surface.

FIG. 5 presents graphically the results of experiments designed to determine the effect on tumor size following treatment with conjugates of the invention.

FIG. 6 shows T cell activity following immunization with conjugates.

FIG. 7 depicts mean volume of tumor following treatment, as compared to controls.

### DETAILED DESCRIPTION OF PREFERRED EMBODIMENTS

### Example 1

As pointed out, supra, soluble MHC molecules are known to the art. A nucleic acid molecule encoding soluble HLA-A*0201 heavy chain was treated to introduce two site directed mutations. Specifically, the codon for glutamic acid at position 275 was mutated to free cysteine, and a stop codon was introduced at position 279, on the C terminal portion of the 3d domain of the heavy chain. Commercially available products, and art recognized methodologies were used in this step. Briefly, however, the expression plasmid pHN1 HLA-A2-BSP, taught by Altman, et al., Science, 274:94-6 (1996), incorporated by reference, was used, in combination with polymerase chain reaction and sequencing methodologies to introduce and to confirm the mutations.

The mutated molecule was then used, together with a nucleic acid molecule encoding 132M, in accordance with Altman, et al, supra, Romero, et al., J. Exp. Med., 188:1641-1650 (1998), and/or Garboczi, et al., Proc. Natl. Acad. Sci. USA, 89:3429-3433 (1992) all of which are incorporated by reference. The expression of the proteins was carried out in E. coli, resulting in inclusion bodies.

The inclusion bodies were refolded, and combined with the known HLA-A2 restricted, immunodominant influenza virus Flu matrix peptide “FLUMA 58-66”, i.e.:

Gly Ile Leu Gly Phe Val Phe Thr Leu (SEQ ID NO:1),

resulting in MHC peptide complexes which were purified on a column.

These monomeric MHC conjugates were used in the further examples which follow.

### Example 2

This example describes the formation of conjugates consisting of a monomeric MHC peptide complex of example 1, and a single murine Fab′ fragment specific to carcinoembryonic antigen, or “CEA.”

Buchegger, et al., J. Exp. Med., 158:413-427 (1983), incorporated by reference, describe murine IgG1 monoclonal antibody 35A7, against CEA. The mAb displays no cross reactivity for antigens expressed by granulocytes.

Monoclonal antibodies were incubated with pepsin, at a 3:100 wt/wt ratio of pepsin/mAb, and incubated at 37° C. in 0.2M acetate buffer, pH 4.0, for 22 hours, to produce F(ab′)₂ fragments. In turn, the F(ab′)₂ fragments were reduced with 10 mM cysteamine, for 1 hour at 37° C., in Hepes/NaCl buffer, pH 7.0, and then separated on a column. This yielded the Fab′ fragments.

In order to conjugate the fragments with the molecules of example 1, the latter were incubated for 2 hours with a 25 molar excess of bismaleimide polyethylene oxide at room temperature, in phosphate buffered saline, pH 7.0. The excess coupling reagent was removed via gel filtration, resulting in 45 kilodalton, bismaleimide derivatized MHC molecules, containing a free thiol group at position 275.

These derivatives were combined, immediately, with a 1.5 molar excess of Fab′ fragments, freshly prepared as described in this example, followed by 18 hours of incubation at 4° C., after concentration of the proteins to 10 mg/ml. Conjugates were purified via FPLC, using commercially available products and known methods, and then analyzed under both reducing and nonreducing conditions, using 10% SDS-PAGE gel electrophoresis.

The conjugates eluted at an apparent molecular weight of about 95 kilodaltons from a molecular sieving column equilibriated in non-denaturing buffer. They showed a major band of about 82 kilodaltons on SDS-PAGE, under non-reducing conditions, apparently due to dissociation of β2M and peptide.

Under reducing conditions, a single band of about 57 kilodaltons was obtained, corresponding to a thioether linked HLA-A2 heavy chain, and a pepsin cleaved, Fab′ heavy chain. The same conjugate could be obtained by derivatizing the mAb with bismaleimide, followed by coupling to the monomeric MHC complex of examples linked HLA-A2 heavy chain and pepsin cleaved Fab′ heavy chain.

### Example 3

This example describes the preparation of additional conjugates. Commercially available antibodies were used. Specifically, HERCEPTIN® is a recombinant, humanized mAb, of human IgG1κ isotype, specific for the extracellular domain of the HER2 receptor. See Carter, et al., Proc. Natl. Acad. Sci. USA, 89:4285-9 (1992), incorporated by reference. RITUXIMAB® is a chimeric, murine/human mAb of IgG1 human κ subtype, directed against the CD20 molecule found on the surfaces of normal and malignant B lymphocytes. See Reff, et al., Blood, 83:435-445 (1994), incorporated by reference.

The same protocol that was used to prepare Fab′ fragments in example 2 was used, with the following exceptions: The HERCEPTIN F(ab′)₂ fragments were incubated with pepsin for 8 hours, and RITUXIMAB was incubated for 15 hours.

Fab′ fragments, and conjugates with monomeric MHC molecules were prepared exactly as described in example 2.

### Example 4

This example describes flow cytometry analyses of the conjugates described in examples 2 and 3, supra.

Various cell lines were used, including LoVo, which is a colon carcinoma cell line that expresses CEA, SK-BR-3, which is a breast carcinoma cell line expressing HER 2 (ErbB2), and B cell lymphomas Daudi and Raji, both of which express CD20. The cells are all commercially available from the American Type Culture Collection. They were cultured in RPMI 1640, supplemented with 10% fetal calf serum. Daudi cells express no MHC Class I molecules, due to deletion of the β2M gene. The other three cell lines are known to be HLA-A2 negative, a fact which was confirmed via assaying with an HLA-A2 specific antibody.

Samples of LoVo, SK-BR3 and Daudi cell lines were incubated with each conjugate, in 50° C.l of PBS, containing 2% BSA, at a concentration of 2 μg/ml for 1 hour at room temperature under gentle agitation. Cells were washed, three times, and then FITC labelled, anti HLA-A2 mAbs were added, and incubated for 30 minutes at 4° C. The cells were washed, twice, and analyzed immediately via FACS. As negative controls, cells which were not incubated with conjugate were used.

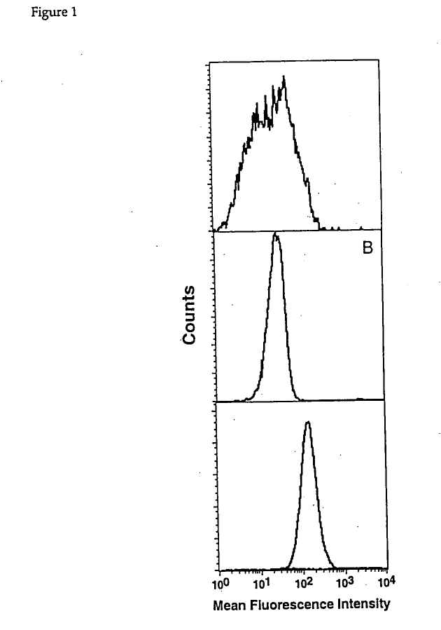

The results as set forth in FIG. 1 showed that, after incubation with the relevant bifunctional conjugates, all three cell lines presented a high density of HLA-A2, indicating that the antibody fragment portion of the bifunctional conjugates had specifically bound to the cell surfaces, and had specifically coated the monomeric MHC/peptide complexes.

### Example 5

These experiments were carried out to determine if CTLs specific for the MHC/peptide complexes would recognize the tumor cells coated with bifunctional conjugates containing a monomeric MHC/peptide complex. Samples of each of the 4 cell lines were incubated for 45 minutes at 37° C., with a 2 μg/ml concentration of monomeric conjugate. The cells were labeled, concurrently, with ⁵¹Cr. Following labelling, cells were washed, three times, with PBS-2% BSA, as described supra, and then 1000 cell samples were incubated, at 37° C. for 4 hours, with an HLA-A2 restricted CTL clone specific for the SEQ ID NO:1/HLA-A2 complex, as described by Valmori, et al., Canc. Res., 59:4050-5 (1999), incorporated by reference. Varying effector: target ratios were used, i.e., 0.1, 1, 10 and 30:1. The cells were incubated in 200 μl DMEM, 10% FCS, in V-bottomed microwell plates. Release of radiolabelled chromium was determined in accordance with Valmori, et al., J. Immunol., 160:1750-8 (1998), incorporated by reference.

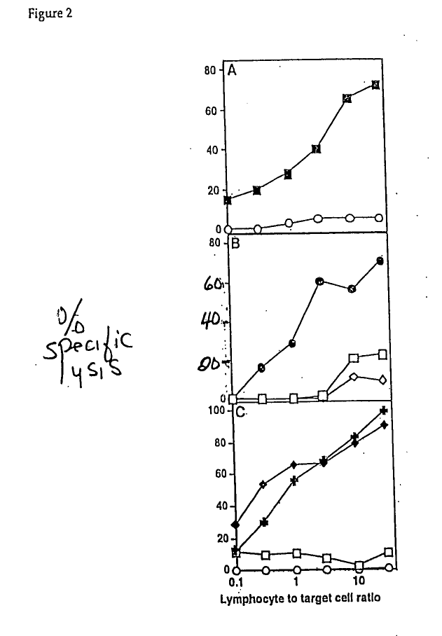

In a first experiment, LoVo cells, which express CEA, were lysed very efficiently by specific CTLs when preincubated with the conjugate containing anti-CEA Fab′ (filled square, panel A), while SK-BR-3 cells, which do not express the molecule, were not (open circles, panel A). These SK-BR-3 cells do express HER2, and were lysed after incubation with the conjugate containing anti-HER2 Fab′ (panel B, filled circles), with minimal lysis of LoVo cells (panel B, open squares) and almost no lysis of Daudi cells (panel B, open diamonds). Daudi and Raji cells both express CD20, and were lysed when incubated the conjugate containing the Fab′ fragment from the CD20 specific mAb (panel C, filled diamonds and crosses), while LoVo and SK-BR-3, which do not express the CD 20, were not (panel C, open squares and circles). In these experiments, the target cells were preincubated with a constant amount of conjugate (2 μg/ml) and the effector to target cell ratio ranged from 1:1 to 30:1. All of these results are set forth in FIGS. 2A-2C.

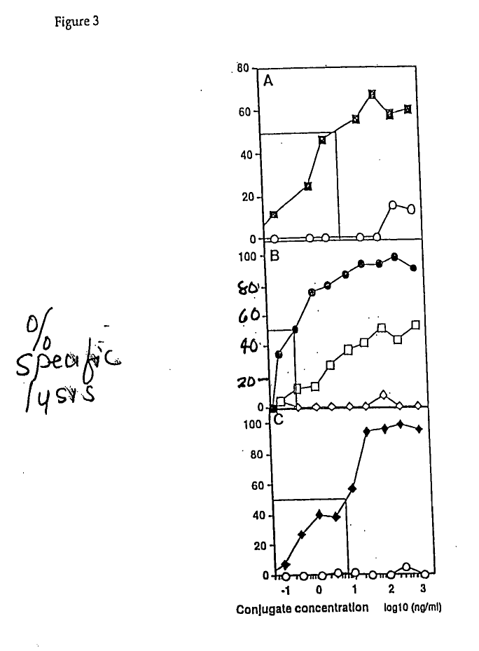

These experiments were continued, in order to titrate the conjugates for determining induction of CTL mediated tumor cell lysis at constant effector/target ratios of 10:1. Increasing concentrations, ranging from 10−¹ to 10³ ng/ml of conjugate, were incubated with the different cell lines described supra, for one hour, at 37° C., after which CTLs were added, and incubated for 4 hours, after which ⁵¹Cr release was measured. The anti-CEA conjugates were incubated with LoVo cells (filled squares), or SK-BR-3 (open circles), in panel A of FIG. 3. In panel B, anti-HER2 conjugates were incubated with SK-BR-3 (filled circles), LoVo cells (open squares), or Daudi cells (open diamonds). In panel C, the anti-CD20 conjugate was used with Daudi cells (filled diamonds), or SK-BR-3 (open circles). In each panel, the conjugate concentration giving 50% specific lysis is indicated. FIG. 3 shows that the conjugate concentration required for 50% lysis ranges from 0.5-8 ng/ml, 5-100 picomolar.

There was one instance where non-specific CTL mediated lysis appeared to occur. LoVo cells express barely detectable levels of HER2, but there was a moderate degree of lysis observed. See FIGS. 2B and 3B, open squares. As such, further experiments were carried out. In these experiments, whole monoclonal antibodies against HER2 (i.e., “HERCEPTIN”) were added, at 20 μg/ml, or not, with increasing concentrations of the anti-HER2 conjugates, to either SK-BR-3 or LoVo cells, and the CTLs described supra.

Unconjugated whole mAb to HER2 inhibited lysis of both SK-BR-3 and LoVo cells to the same degree, confirming that the lysis was due to the specificity of the antibody fragment of the conjugate indicating that the moderate degree of lysis was specific, probably due to low expression of HER-2 on LoVo cells. What was also observed was that, notwithstanding 20 μg/ml of competing mAb, the conjugates were still able to stimulate maximal lysis of SK-BR-3 at concentrations of 1 μg/ml or higher, confirming the high potency of the conjugate.

The results demonstrate that a bifunctional conjugate containing a monomeric form of MHC viral peptide complex and a single monovalent anti-tumor marker antibody fragment can induce very efficient and sensitive lysis of epithelial and lymphoid cancer cells by viral specific cytotoxic T lymphocytes. Cells coated by the antibody Fab′ fragment, monomeric MHC/viral peptide complexes are lyzed as efficiently as if they were infected by the relevant virus.

### Example 6

These experiments were designed to determine the ability of the conjugates described supra, to mobilize intracellular Ca²⁺ in specific CTLs. See Valitutti, et al., J. Exp. Med., 181:577-584 (1995), incorporated by reference for a discussion of the phenomenon of Ca²⁺ mobilization in CTLs following T cell activation in the specific CTL clone.

The same assay as described supra was carried out, and Ca²⁺ mobilization following incubation with anti-HER2-Fab-HLA-A2/Flu conjugates, on SK-BR 3 cells, was studied.

Overall levels of mobilized Ca²⁺ observed following incubation with conjugate coated, SK-BR-3 cells, was comparable to that obtained with standard, anti-CD3 cross linking.

In contrast, the same anti-ErbB2 HLA-A2 Flu conjugate in soluble form without the target cells, did not induce specific T cell activation. Thus, oligomerization of Fab-HLA-A2/Flu conjugates as a result of binding to cell surface to tumor antigens was shown to play an essential role on CTL activation.

### Example 7

This example, and the examples which follow, describe the preparation and use of complexes which consist of Fab′ fragments, and streptavidin, conjugated to streptavidin/botin-MHC-peptide tetramers. Fab′ fragments from the antibodies described supra were used. The Fab′ fragments were conjugated to streptavidin by incubating a five molar excess of reduced Fab′ with streptavidin that had been derivatized with 4-8 mol of maleimede, for 16 hours, at 4° C., in 50 mM sodium acetate, 0.5 mM EDTA buffer, pH 7.0. The resulting Fab′-streptavidin conjugates were purified via FPLC. They eluted with an apparent molecular weight of 150-200 kilodaltons, suggesting 2-3 Fab′ molecules were coupled per streptavidin molecule. The streptavidin molecules conjugated to Fab′ fragments were used to assemble tetramers of biotinylated MHC/peptide complexes.

In brief, purified HLA-A*0201 heavy chain and β2M molecules were synthesized, using a commercially available prokaryotic expression system, using well known methodologies. The heavy chain was modified by deleting the transmembrane cytosolic tail, and the C-terminal addition of a sequence containing the BirA enzymatic biotinylation site. The heavy chain, β2M, and the peptide of SEQ ID NO:1 were refolded by dilution. The molecular weight of the desired product was 45 kilodaltons. Such products were isolated via FPLC, and then biotinylated in the presence of biotin, adenosine 5′-triphosphate, and Mg²⁺.

Following this, either Fab′ streptavidin conjugates, or free streptavidin was incubated, for 1 hour at 4° C., with the biotinylated, HLA-A*0201/peptide complexes, in a 1:4 molar ratio, and then concentrated to 1 mg/ml. See Altman, et al., Science, 274:94-92 (1996); Romero et al., J. Exp. Med., 188:1641-1650 (1998), incorporated by reference.

Three complexes, corresponding to the antibodies discussed supra, were made, i.e.:

anti-CEA-Fab-SA-A2/Flu

anti-HER2-Fab-SA-A2/Flu

anti-CD20-Fab-SA-A2/Flu

The conjugates of Fab, streptavidin, HLA-A*0201 and peptide eluted on FPLC at an apparent molecular weight of 350-400 kilodaltons, which suggests full tetramerization of the MHC on Fab′-streptavidin conjugates.

### Example 8

These experiments describe the capacity of the Fab′-SA-MHC tetramers conjugates to coat HLA-A2 onto the four, HLA-A2 negative cell lines described supra. The cells were incubated with the conjugates in 20 μl of PBS-2% BSA, at a concentration of 100 μg/ml. After washing, the cells were incubated with FITC labelled, anti-HLA-A2 antibodies, as described supra, for an additional 30 minutes. The cells were washed, twice, in the same buffer, and analyzed immediately via FACS.

The colon carcinoma cell line LoVo, which is positive for CEA, gave a positive signal when preincubated with anti-CEA-Fab-SA-HLA-A2/Flu conjugate, but was negative when preincubated with anti-HER2-Fab-SA-HLA-A2/Flu. In similar fashion, SK-BR-3 and the Raji and Daudi lines gave a positive signal only when preincubated with either anti-HER2-Fab-SA-HLA-A2/Flu or anti-CD20-Fab-SA-HLA-A2/Flu.

Following these experiments, titration assays were carried out, using concentrations of conjugate ranging from 3-200 μg/ml, under the same conditions.

The anti-ErbB-2-Fab-SA-HLA-A2 conjugate was the most potent, probably the result of the high affinity of the source antibody.

### Example 9

These experiments describe the results of cell lysis assays carried out using the Fab′-SA-HLA-A2/Flu conjugates described, supra. The four cell lines described supra were used as targets. Samples of each cell line were incubated for 2 hours, at room temperature, with each of the different conjugates, at concentrations of 40 μg/ml. The cells in the samples were then washed three times, with PBS-BSA, and then labelled cells (1000 cell samples), were incubated with the CTL described supra, at effector:target cell ratios ranging from 0.1 to 30. Incubation took place in 200 μl of DMEM, 10% FCS, in V-bottom microwells, in the presence of 3 μg/ml human β2M. Chromium release was calculated as described supra.

As a negative control, ⁵¹Cr labelled target cells were preincubated with streptavidin-A2/Flu tetramers, without Fab, or an irrelevant Fab fragment, and tested with the same CTL.

### Example 10

These experiments describe the manufacture of a soluble mutant MHC complex, combined with a peptide. An H-2K^{b} heavy chain construct, with a mutant cysteine at its C terminus, was used. See Kalergis, et al., J. Immunol. Meth., 234 (1-2): 61-70 (2000), incorporated by reference. This molecule was modified further, by site specific mutation, to replace wild type cysteine, at position 121, with alanine.

Modified H-2K^{b} molecules, and β2M molecules, were produced as inclusion bodies in E. coli, following Kalergis, et al., supra, renatured and refolded, together with chemically synthesized, H-2K^{b} restricted ova²⁵¹⁻²⁶⁴ peptide, i.e., SINFEKL (SEQ ID NO: 1). The entire complex was refolded via dialysis, and purified on a Sephacryl S100 column. This complex presents a free thiol group at its C terminal cysteine residue.

The mAb referred to as 35A7, is a murine antibody class IgG1 and is described by Buchegger, et al., J. Exp. Med., 158(2):413-427 (1983). It binds to carcinoembryonic antigen (CEA) and does not bind to cross reacting antigens expressed by granulocytes. F(ab′)₂ fragments of 35A7 were prepared by digestion with pepsin, at a 3:100 (wt/wt) ratio of pepsin/IgG1, and were then incubated, at 37° C. in 0.2M acetate buffer, pH 4, for 15 hours, followed by gel filtration. Fab′ fragments were then obtained by reducing the F(ab′)₂ fragments with 5 mM beta-mercaptoethanol for 30 minutes at 30° C., in 0.15 mM phosphate buffer, pH6.5 followed by gel filtration in accordance with Glennie, et al., J. Immunol., 139(7):2367-2375 (1987).

The Fab′ fragments were then incubated with a 25 molar excess of N—N-ortho-phenylene dimalemide (o-PDM), for 2 hours at room temperature in 0.15M phosphate buffer, pH 6.5. Excess o-PDM was removed by gel filtration. This results in Fab′ fragments in which cysteine groups have been derivatized, having a molecular weight of about 50 kDa.

### Example 11

The two materials, i.e., the complex, and the Fab′ fragment discussed in example 10, supra, were concentrated to 1 mg/ml, and a 1.5 molar excess of the bismaleimide derivatized anti-CEA Fab′ fragments were mixed with freshly prepared, H-2K^{b}/ova peptide complexes, followed by 2 hours of room temperature incubation, leading to formation of thioether bonds between the free thiol group referred to supra, and the derivatized Fab′ fragments. These will be referred to as “conjugates” hereafter. Sometimes, “CEA-Fab′-H-2 Kb will be used interchangeably with “conjugates.” Conjugates were purified by FPLC on a Superdex 200 column, and were analyzed, under both reducing and non-reducing conditions (12% SDS-PAGE).

The major fraction eluted at an apparent molecular weight of 95 kilodaltons. About 5% of refolded H-2K^{b} manomer was obtained, and the coupling frequency was about 30%.

### Example 12

The purified complexes of H-2K^{b}, β2 microglobulin and the peptide, the Fab′ fragment, and the conjugate of Fab′ and complex were all analyzed via standard SDS-PAGE analysis, under reducing and non-reducing conditions.

Under non-reducing conditions, the complex (45 kDa) and Fab′ fragment (50 kDa), when conjugated, migrate with a major band of 83 kDa, and a discrete, minor one at 12 kDa, which represents dissociated β2 microglobulin. When reduced, dissociation of the two chains from the Fab′ fragments is partial, because of some covalent throether bonds created after reduction, and the bismaleimide treatment.

The MHC complex migrated in a similar fashion at both reducing and non-reducing conditions. There is a major band at 58 kDa, which corresponds to dissociation of β2 microglobulin and Fab′ light chains. Due to the covalent binding mentioned supra, there is a second band, at 83 kDa.

### Example 13

This example describes experiments designed to test the capacity of the conjugate to bind to a CEA expressing cell line, i.e., H-2K^{b} negative, human carcinoma cell line “LoVo” which is available from the American Type Culture Collection, as ATCC CCL-229. This cell line was maintained in culture in RPMI medium, supplemented with 10% fetal calf serum.

Samples of the cell line were incubated with the conjugate, described supra, at a concentration of 10 μg/ml, for 45 minutes, at room temperature, in 50 μl of PBS, 2% BSA, 0.02% azide. The cells were washed, twice, and then conjugates were analyzed via FACS, using a conformation sensitive anti-H-2K^{b} monoclonal antibody labeled with FITC that was added to the cells for 20 minutes, at 4° C. Intact, anti-CEA mAb 35A7, at a concentration of 10 μg/ml, followed by an anti-mouse IgG Fc specific-FITC conjugate, was used as a positive control. These assays permit determination of the proper conformation of the refolded H-2K^{b} molecules, as well as binding.

The results, demonstrated that the LoVo cells were coated efficiently with conjugate, and that the refolded molecule had a native conformation.

### Example 14

Two strains of transgenic mice were used, i.e., OT-1 TCR transgenic mice, of C57BL/6 background, which are described by Hagquist, et al., Cell, 76(1):17-27 (1994), and CEA transgenic mice [C57BL/6J-IgN (CEAGe)18FJP], described by Clarke, et al., Cancer Res., 58(74):1469-1477 (1988).

CTLs specific for complexes of H-2K^{b} and the peptide of SEQ ID NO: 1 were generated from spleen cells of an OT-1 mouse, which had been incubated, for five days, with 1 μM of ova peptide, using standard methods.

Two target cells were used, i.e., murine CEA transfected carcinoma cell line, and CEA expressing human carcinoma cell line LoVo. These were used to test the capacity of the CTLs to lyse targets. The murine line was incubated for one hour, at 37° C., with an irrelevant VSV peptide, and then it and the human cell line were incubated, for 45 minutes, with 10 μg/ml of the anti-CEA-H-2K^{b} ova conjugate referred to supra. All cells were labeled with ⁵¹Cr during this incubation.

Cells were washed, three times, with DMEM, and then 2000 target cells/well were incubated, for 4 hours, at 37° C., with differing ratios of CTLs. Chromium release was measured, and the percentage of specific lysis was calculated as:

$$100 \times [ \frac{[ ( experimental - spontaneous release ) ]}{( total - spontaneous release )} ]$$

As a negative control, C15 tumor target cells were used that had been preincubated alone with the irrelevant peptide, as were LoVo cells without complex, tested with the CTL clone.

The CTL cells, when coated with the complexes, were specifically killed, with 45% lysis obtained at a 90:1 ratio. No killing was observed in the absence of conjugate. Similarly, up to 40% lysis of the human clones were observed, at a 30:1 ratio, showing that coating target cells with the complex were susceptible to T cell lysis.

### Example 15

These experiments relate to the localization of the conjugates in mice, so that the capacity of intravenously injected conjugates in target mice could be evaluated.

Three, CEA transgenic mice, and four nude mice of Swiss genetic background, were grafted, subcutaneously, with either CEA transfected colon carcinoma MC38-C15, or in the case of the nude mice, with human colon carcinoma LS174T, which expresses CEA on one flank and MC38-C15, on the other.

A total of 20 μg of purified conjugates was labeled with 20 μCi of ¹²⁵I, following standard methods. Size of conjugate was controlled via filtration on FPLC, where it eluted at 95 kDa, the same size as unlabeled material. The labeled material and percentage of immunoreactivity was determined via a 3 hour incubation, at room temperature, using an excess of CEA that had been coupled chemically to CNBr Sepharose. It was found to be 70-80%.

Following this, 2 μg of the conjugates, labeled with 2μ Curies of the ¹²⁵I, were injected, intravenously, into the mice, via the tails. The injections were given when tumor mass reached a volume between 50 and 300 mm³.

Twenty four hours later, the mice were sacrificed, tumor and normal tissues were dissected, weighed, and radioactivity measured.

The results showed that the ¹²⁵I labeled conjugates specifically targeted the C15 and LS174T tumor grafts.

As a follow-up to these experiments, F(ab′)₂ fragment from a mAb specific to EGF receptor, and one with irrelevant specificity were labeled with 20 μCi of ¹³¹I, and one of each was co-injected with the ¹²⁵I labeled conjugate, into the tail veins of the mice. The F(ab′)₂ of irrelevant specificity was used in the CEA transgenic mice, while the anti-human EGF receptor specific F(ab′)₂ fragment was used in the nude mice. Radioactivity of both isotopes was measured in a dual channel scintillation counter, and showed that the EGF receptor specific fragment targeted the human xenograft only, showing the specificity of the anti-CEA-Fab′-H-2K^{b} conjugate. The irrelevant antibody did not target at all. These results confirm the in vivo targeting specificity.

### Example 16

The OT-1 transgenic C57BL/6 mice, referred to supra, carry TCR genes (Vαx2, Vβ5.1), isolated, from a T cell clone that is reactive with SEQ ID NO: 1. See Holquist, et al., Cell, 76(1):17-27 (1994). More than 90% of CD3 positive cell express this transgene. This was useful for the experiments which follow, designed to determine if the conjugates inhibited tumor growth.

First, 7.5×10⁵ syngeneic colon carcinoma cells 200 μl (C15 cells), which express CEA were subcutaneously grafted into 10, OT-1 mice, using PBS. One group of five mice received six, systemic injections of 20 μg of the conjugate, described in example 15, supra at days 1, 4, 8, 18, 21, and 24. A control group of the other 5 mice was injected in parallel with 20 μg of F(ab′)₂ fragments from the anti CEA mAb, the first injection was intravenous, the rest intraperitional without conjugate. Tumor size was measured every two days.

There was a significant delay in tumor growth in the conjugate treated mice. By day 25, two of the conjugate treated mice had no detectable tumor, one had only a tiny nodule, and two had small tumors, not exceeding 20 mm³. In the controls, all tumors ranged from 120 to 320 mm³.

There was no overlap in standard deviation between the groups. For example, at day 28, the mean tumor size for the treated group was 77 mm³, and 633 mm³ for controls.

In follow ups, three of five mice in the conjugate groups did not develop tumors, while as all 5 in the control group, developed large tumors, after 30 days.

### Example 17

These experiments involve studies on tumor regression and growth inhibition, following adoptive transfer. The approach described herein permits investigation via immunization and active stimulation in a normal environment close to actual cancer immunotherapy conditions. Further, the transgenic mice are less likely to produce anti-CEA antibodies or T cells directed against CEA expressed by the graft.

Ten unirradiated CEA transgenic mice were adoptively transferred, via intraperitoneal injection with 50 million OT-1 cells which had been harvested and washed, as described supra, and were immunized, one day later, with 200%1 g of full length ovalbumin in Montanide adjuvant. Twenty days after the adoptive transfer, they were subcutaneously grafted with 106 C15 cells, in 200 μl PBS. The late grafting excludes non-specific tumor growth inhibition caused by cytokine release during the peak immune reaction. After 8 days, tumor nodules were palpable in all animals. Five received an intravenous injection of 20 μg of the conjugate in 200 μl of PBS, and then intraperitoneal injections of the same materials every two days. The remaining mice, which served as a control, received 20 μg of anti-CEA F(ab′)₂ fragments without MHC, in the same volume of PBS. At 20 days following the graft, all 10 mice received a boost of 100 g ovalbumin.

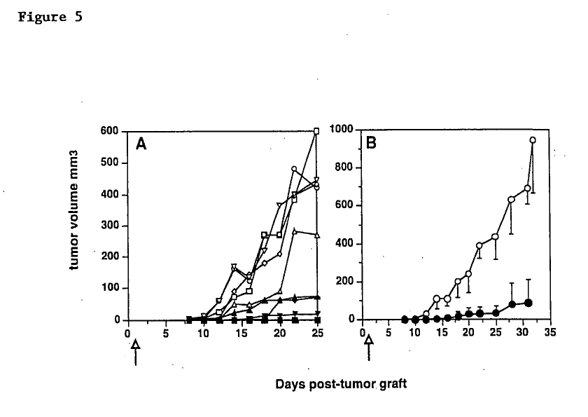

Tumor growth was monitored every two days, with length, width, and height being measured with calipers. Size was expressed as (length×width×height)/2. Mean tumor size and standard deviation were calculated for each group. FIG. 5 depices these results in graphic form.

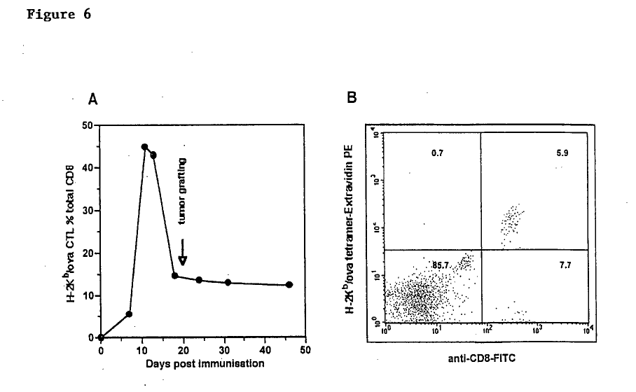

Peak activity of the specific T cells, as can be seen in FIG. 6, was reached 10 days after immunization, and the frequency ranged between 12 and 40%.

Results taken 24 days after the tumor graft indicated that four of the five mice that had been treated with conjugates had tumor volumes below 20 mm³. All mice in the control group had tumors larger than 200 mm³, as did one conjugate treated mouse.

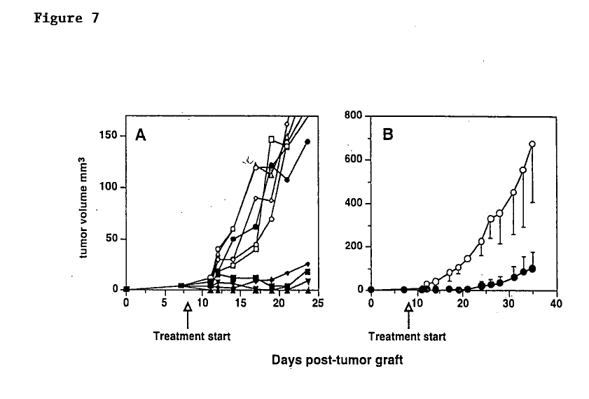

Thirty-six days after the tumor graft, when the tumor which escaped therapy is excluded, mean volume of conjugate treated tumors was 100 mm³, as compared to 676 mm³. Even if the excluded tumor is added, the average size is 227 mm³. These results, depicted in FIG. 7 thus indicate the anti-CEA-Fab-H-2K^{b} ova conjugates can induce tumor growth inhibition, even when tumors are established and palpable.

### Example 18

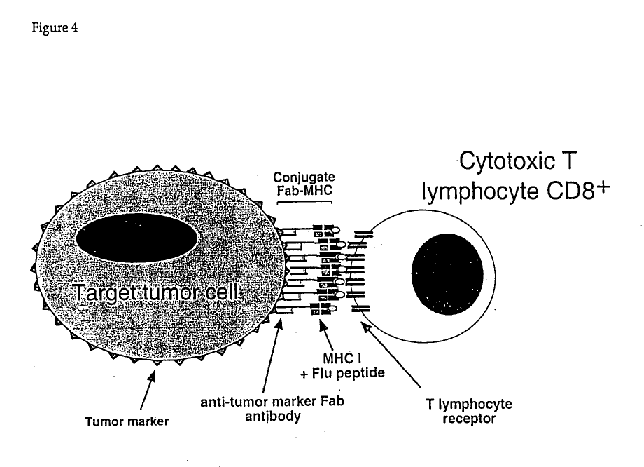

The mechanism by which the Fab′ antibody fragments conjugated to monomeric MHC/peptide conjugates induce efficient tumor target cell lysis is schematically described in FIG. 4. The key feature is that the bifunctional conjugates induce the binding and subsequent activation and cytolytic activity of CTLs, but only when they are oligomerized on tumor cells expressing a high density of tumor-associated antigen. Individual, soluble conjugates cannot bind and activate the specific CTL as shown in Example 6, due to the known low affinity of individual MHC complexes for the T cell receptor. See Altman et al., Science, 274:94-6 (1996) incorporated by reference.

This has important implications for clinical use of the described bifunctional conjugates, i.e., when intravenously injected, the above-described conjugates with monomeric MHC/peptide complexes will not activate specific T lymphocytes in the circulation. The bifunctional conjugates will be oligomerized on the cell surface, only when they have reached a tumor cell with high enough density of antigen or differentiation marker. Then, by cooperative binding, the oligomerized MHC/peptide complexes activate cytotoxic T lymphocytes and induce target cell lysis. Thus taking the old analogy of antibodies as guided missiles, here the missiles will be fired only when they reach their targets. Further, as a consequence of the absence of T cell activation by soluble bifunctional conjugates, such conjugates will be much less toxic than, for instance, the antibody-superantigen conjugates described by Dohlstein et al., Proc. Natl. Acad. Sci. USA, 88:9287-9291 (1991), and Giantonio et al., J Clin. Oncol. 15:1994-2007 (1997), incorporated by reference. Indeed, in the experiments described, supra, when the Fab′-H-2K^{b}-ova conjugates were injected into OT-1 mice, no toxicity was observed, even though 90% of T cells of such mice are specific for this complex. Thus, the bifunctional conjugates described here can be injected in large enough amounts to target all accessible tumor cells. Still further, normal cells which may express small copy numbers of tumor-markers will not induce the CTL binding and lytic activity. This is significant, since numerous tumor markers or tumor associated antigens are know which are recognized by antibodies, and are abundant on cancer cells, but present at low densities on normal cells. Another practical advantage of conjugate or fusion protein made of single antibody fragment and monomeric MHC/peptide complex is that their relatively small size 95 kDa for instance for the conjugate described in Example 1, supra. This size is optimal for in vivo tumor targeting as shown for F(ab′)₂ fragments of 100 kDa from anti-CEA monoclonal antibodies in experimental and clinical studies. Buchegger et al., J Exp. Med., 158:413-427 1983 and Delaloye et al., J Clin. Invet., 77:301-311 1986 (for review, see Mach in Peckham M., Penedo, H. and Veronesi, U., Oxford Textbook of Oncology, Vol. 1, Oxford University press, pp-81-103 1995, incorporated by reference.

The foregoing disclosure sets forth the aspects of the invention, which relates to conjugates of formula
A-B—(C)n
wherein A is a specific binding protein, B is optional and, when present, is a binding partner to which both “A” and “(C)n” bind, “C” is a MHC combining a heavy chain of, e.g., a specific HLA molecule, a β₂M molecule, and a peptide, and “n” is a whole number, which preferably ranges from 1 to 10, most preferably 1, when A is a Fab′ fragment, and B is absent.

It is preferred that “A” is an antibody or a binding portion of an antibody, such as a Fab′ fragment or an F(ab′)₂ fragment or a single chain Fv fragment. The antibody or binding fragment is chosen so as to create a conjugate that binds specifically to an antigen, such as a cell surface tumor-associated or differentiation marker. The examples given supra, i.e., CD20, CEA, A33, Erb2, and HER2, are exemplary, but are by no means the only examples, of cell surface molecules to which the specific binding protein may be directed. For example, antibodies and antibody fragments which bind to antigen A33 (colon cancer), G250 (renal cancer), 3S193 (anti-Lewis Y antibody), effective against epithelial cancers, such as carcinomas, breast, epithelial, colon, and lung cancer, “806,” which is a ΔEGFR specific antibody useful against glioblastoma, sqauamous cell carcinoma, head and neck cancer, and non-small cell lung cancer, and I<M871/KWL871, which is effective against melanoma, can all be used.

Whereas antibodies and binding fragments of antibodies are preferred, other binding proteins can be used. For example, the binding of receptor molecules and their specific ligand is well known. This specific binding arrangement can be exploited in preparation of the conjugates of the invention, such that “A” may be a ligand or receptor molecule, or a portion of such molecules known to be involved in receptor/ligand interaction. Exemplary of such interactions is that between epidermal growth factor (EGF) receptor and EGF and others are well known, and need not be repeated here.

As “B” is an optional part of the conjugates of the invention, it will be discussed infra. Attention now turns to “C”, which comprises an MHC/peptide complex. As was explained, supra, MHC molecules contain a heavy chain, a β₂M molecule, and a peptide. Polymorphisms result in a wide variety of different types of MHC molecules, such as HLA-A1, A2, B27, Cw6, etc. These are all so-called “Class I” molecules. The art will also be familiar with “Class II” molecules, such as HLA-DR, and so forth. Any of these varieties of molecule may be used in the conjugates of the invention.

The MHCs also contain a peptide. As is well known in the art, the peptides which are a part of MHCs can, and do take various forms. Depending upon the nature of the HLA molecule, the nature of the peptide will change. There are various ways to choose the peptide which is used in the MHCs, such as using motif analysis, as described by Rammensee, et al., Immunogenetics, 41:178-228 (1995); Ruppert, et al., Cell, 74:929-937 (1993); Hunt, et al., Science, 255:1261-1263 (1992); Falk, et al., Nature, 351:290-296 (1991) all of which are incorporated by reference.

Exemplary of the complexes which could be used are complexes which include defined, viral epitopes which the act recognizes as generating specific memory responses against a virus. Vaccines are known for some of these viruses. Combinations of complexes as defined herein and vaccines can be used in a vaccination schedule where a defined, specific immune response is desired. A by no means inclusive listing of these complexes includes complexes of HLA-A1 molecules and either the influenza A basic polymerase I 591-599 peptide (numbering refers to positions within the protein), or influenza A nucleoprotein 44-52. When HLA-A2 molecules are the MHC molecule in question, the peptide partner can be, e.g., HBV envelope protein HBs 251-259, HBV pol 816-824, EBV LMPZA 426-434, EBV EBNA3C 284-293, HBV pol 773-782, influenza B nucleoprotein 85-93, or influenza A matrix protein 58-68. When HLA-A3 is used, exemplary peptides include EBV EBNA 3A 603-611, and influenza A nucleoprotein 265-273. Indeed, an immunerable number of complexes can be determined by review of, e.g., Marsh, et al., The HLA Facts Boos, (Academic Press, 2001), incorporated by reference in its entirety.

For inducing CTL lysis of cells, such as tumor cells, the conjugates may contain immunodominant, viral peptides, against which the patient has an active T cell memory repertoire. In the alternative, if the patient has an active T cell response against defined tumor rejection T cell antigens, the specific peptides involved in the response may be used. Another option is to use peptides known to be recognized by alloreactive T lymphocytes. A treatment protocol for a cancer patient using bifunctional antibody Fab-MHC/peptide conjugates in accordance with the invention, may include, e.g.:

A) HLA typing of the patient;

B) Analysis of the patient T cell repertoire against immunodominant common virus peptides, such as CMV, EBV or influenza viruses, restricted to his or her own MHC, as well as, in certain cases against the patient's own tumor rejection peptide antigens recognized by T lymphocytes;

C) Identification of the tumor markers, or tumor associated antigens or differentiation markers expressed more abundantly by the patient's tumor cells and recognized by available monoclonal antibodies;

D) Selection of the monoclonal antibodies according to the result of the analysis of point C and preparation of Fab′ fragment according to the invention;

E) Preparation of soluble MHC compatible with the patient HLA typing as described in supra, containing the MHC restricted most antigenic peptides selected according to analysis as above;

F) Synthesis of the Fab-HLA/peptide conjugate according to the invention;

G) Administer a booster of vaccination with the live virus from which the antigenic peptide was selected for making the conjugate, or a repeated course of active peptide immunotherapy with the selected tumor rejection antigenic peptide;

H) A few days after vaccination, boost or repeated peptide immunotherapy. The patient receives several intravenous injections of increasing doses of the bifunctional Fab-MHC/peptide conjugates;

I) Following in vivo targeting of the injected Fab-MHC conjugate on the tumor cells in vivo and the patient specific T lymphocytes lyse the MHC/antigenic peptide coated cancer cells, as if they were specifically injected by an antigenic virus.

When “B” is not present in the complexes, “A” and “C” may be prepared via the use of e.g., nucleic acid coding constructs which encode fusion polypeptides. Such techniques are well known, as is described, supra. One may also modify the elements “A” and “C” to connect them chemically, as was shown in the examples. One may add amino acid sequences such as those found in the Jun and Fos oncogenes, which then bind A and C via leucine zipper formation. Other alternatives are available, which the skilled artisan will note.

When “B” is used, this comprises a molecule or molecules which facilitates the linking of “A” and “C.” B can also comprise a specific binding pair of molecules, or a complex thereof, such as a complex of avidin or streptavidin or a chemically modified form of streptavidin or avidin, and anywhere from 1 to 4 biotin molecules. For example, B can be a bispecific antibody with one arm directed against a “Tag” epitope placed at the C terminus of A, and the other arm directed against another “Tag” epitope placed at the C terminus of C. The number of binding antibody fragments may vary. Preferably, from 1-5 are used. One may also use, e.g., a bifunctional antibody, or any other molecule or molecular complex to which “A” and “C” can both be joined such as an additional antibody, or binding fragment of an antibody. In particular, an additional antibody fragment which has the property of activating the T lymphocytes, such as anti-CD-28 antibody or a recombinant ligand, such as B7.1, B7.2, or IL-2 for a receptor that activates T lymphocytes, may be used. These additonal materials may be linked to a free cysteine, residue on the first Fab′ fragment from the Fab-MHC conjugate. The use of free cysteine on a bispecific antibody to synthesize trispecific antibodies is taught by Tutt, et al., J. Immunol., 147:60-69 (1991), incorporated by reference. If fusion proteins are used, then a single cysteine residue allowing the coupling of the T lymphocyte activating, third molecule, can be introduced via, e.g., site specific mutation between the two partners of the fusion protein.

With respect to “n,” this will vary depending upon the nature of the other elements of the complex. When “B” is used, for example, there is potential for four biotin molecules, and each biotin molecule can be used to bind an MHC molecule. In such a case, “n” may range from 1 to 4, and is preferably 4. If “B” is an antibody, it can bind two molecules of “C,” and hence “n” will be “2.” The art is familiar with how to determine the number of elements in the conjugate.

The conjugates of binding partners and HLA/β2 microglobulin/peptide may be labelled, using any of the labels known to the art, so as to monitor binding to target cells, to determine the number of bound conjugates, and to establish relationships between these values and the triggering of specific T lymphocytes. Examples of labels include enzymatic labels, such as alkaline phosphatase, metal particles, colored plastics made of synthetic materials; radioactive labels fluorescent labels, etc. Any of these may all be used.

The conjugates may be used, e.g., to identify or to isolate cytolytic T cells present in a sample, where these cells are specific for the HLA/β2 microglobulin/peptide complex. As the examples show, such cytolytic T cells bind to the immunocomplexes of the invention. In a preferred embodiment, the sample being tested is treated with a reactant which specifically binds to a cytolytic T lymphocyte of different phanalyne, wherein said label provides a detectable signal. The sample, including labelled CTLs, is then mixed with target cells coated with conjugates labeled with a fluorochrome. Labelled lymphocytes bind conjugate coated target cells, forming cell clusters which can be separated, preferably by FACS, or by any of the standard, well known approaches to cell separation, such as magnetic cell sorting or density gradient centrifugation. Another separation method can be incubation of the T lymphocyte samples with immobilized target cells coated with different Fab′-MHC/peptide complexes. The peptide used may be chosen by the skilled artisan, depending upon the nature of the specific MHC system under consideration.

Additionally, the method can be used to monitor the status of lymphocyte reactivity against tumors, following administration of a particular therapeutic agent, such as a vaccine. Functional T lymphocyte activation and cytolytic tests can be performed on patient T lymphocytes incubated with target cells coated with bifunctional Fab′-MHC/peptide conjugates. The use of, e.g., Daudi cells devoid of MHC Class I molecules as a target, permits the artisan to determine, e.g., the number of anti-CD20 Fab′-MHC Class I with different peptides, such as TRAs, necessary for inducing patient T lymphocyte activation and cytotoxicity. Further, the methodology can be used to identify cytolytic T cell precursors.

Also a part of the invention is the use of conjugates as described, in conjunction with other steps, to yield populations of T cells with desired features, such as specificity and phenotype. These include distinct cell surface phenotypes associated with antigen experienced, or memory cells, or naive cells, and so forth. Such populations can be cultured, in the presence of either bifunctional Fab′-MHC/peptide conjugate coated target cells, or free peptides in target cells to determine to what extent the deletion of MHC molecules are responsible for the lace of reactivity of T lymphocytes for CTLs. This culturing can be carried out with a mitogen such as phytohemagluttinin, e.g., without peptides, for comparison.

The invention also involves methods for obtaining desired T cells via in vitro or by in vivo recruitment, using the same type of bifunctional conjugates in which the antibody or binding protein is directed against a surface marker expressed by “antigen presenting cells.” In this case, one can predict that, by a similar oligomerization of MHC on target cells, as described in FIGS. 1 and 4 supra, the targeted antigen presenting cells will stimulate the activation and proliferation of functionally naive T lymphocytes specific for the peptide associated with the bifunctional conjugates. This presentation of selected peptide can be used to improve vaccinations approaches.

The terms and expressions which have been employed are used as terms of description and not of limitation, and there is no intention in the use of such terms and expressions of excluding any equivalents of the features shown and described or portions thereof, it being recognized that various modifications are possible within the scope of the invention.

We claim:

1. A conjugate or fusion protein of formula:
A-B—(C)n
wherein A is a protein or polypeptide which binds specifically to a target cell surface, B is optionally present, and comprises at least one molecule which binds to both A and (C)n, C is an MHC/peptide complex, and n is a whole number ranging from 1 to 10.

2. The conjugate of claim 1, wherein B is absent, and n is 1.

3. The conjugate of claim 2, wherein A is an antibody or a binding fragment of an antibody.

4. The conjugate of claim 4, wherein A is an Fab′ fragment of an antibody.

5. The conjugate of claim 2, wherein A is a single chain antibody.

6. The conjugate of claim 4, wherein C is a single chain MHC complex.

7. The conjugate of claim 5, wherein C is a single chain MHC complex.

8. The conjugate of claim 1, wherein A is an antibody, a ligand which binds to an antigen, or a ligand which binds to a differentiation marker overexpressed in tumor cells.

9. The conjugate of claim 1, wherein B is present.

10. The conjugate of claim 5, wherein B comprises a streptavidin or avidin molecule, and from 1 to 4 biotinylated MHC molecules.

11. The conjugate of claim 10, wherein B comprises a streptavidin molecule and 4 biotin molecules.

12. The conjugate of claim 11, wherein A comprises an antibody binding fragment.

13. The conjugate of claim 11, wherein A is an Fab′ fragment.

14. The conjugate of claim 1, wherein said MHC molecule comprises a tumor rejection antigen.

15. The conjugate of claim 1, wherein said MHC molecule comprises an antigenic, viral peptide.

16. The conjugate of claim 2, wherein A is a ligand which binds to a receptor.

17. A method for alleviating cancer in a subject in need thereof, comprising administering to said subject an amount of the conjugate of claim 1 sufficient to bind to cancer cells in said subject and to provoke a T cell response against said cancer cells.

18. The method of claim 17, wherein A is an antibody or a binding fragment of an antibody.

19. The method of claim 18, wherein A is a Fab′ fragment of an antibody.

20. The method of claim 18, wherein A binds to a tumor associated antigen or to a differentiation antigen found on tumor cells.

21. The method of claim 18, wherein A binds specifically to carcinoembryonic antigen.
